(19) United States
(12) Patent Application Publication
(10) Pub. No.: US 2013/0259948 A1
(43) Pub. Date: Oct. 3, 2013

(54) SPRAY DRIED POWDER FORMULATION FOR VACCINES ENTRAPPING ALUM AND THE ANTIGEN IN BIODEGRADABLE POLYMER PARTICLES

(71) Applicants: Amulya Kumar Panda, New Delhi (IN); Anish Chakkunkal, New Delhi (IN); Dinesh Giri Goswami, New Delhi (IN)

(72) Inventors: Amulya Kumar Panda, New Delhi (IN); Anish Chakkunkal, New Delhi (IN); Dinesh Giri Goswami, New Delhi (IN)

(73) Assignee: NATIONAL INSTITUTE OF IMMUNOLOGY, New Delhi (IN)

(21) Appl. No.: 13/824,864

(22) PCT Filed: Sep. 20, 2011
(86) PCT No.: PCT/IB2011/002173

§ 371 Date: Jun. 14, 2013

(30) Foreign Application Priority Data
Sep. 21, 2010 (IN) 2256/DEL/2010

(51) Int. Cl. A61K 39/39

(52) U.S. Cl. CPC A61K 39/39
USPC 424/497; 424/490; 424/184.1; 424/244.1; 424/247.1

(57) ABSTRACT

The present invention relates to a novel effective dry powder vaccine formulation that increases the immune response in the host. The formulation comprises of an antigen entrapped into a polymer particle, coated with alum, finally spray dried into a dry powder. This formulation is used to elicit long lasting higher antibody titers than alum adsorbed antigen or admixture of polymer entrapped antigen and alum.

### FIELD OF THE INVENTION

The present invention relates to a novel effective dry powder vaccine formulation that increases the immune response in the host. The formulation comprises of an antigen entrapped into a polymer particle, coated with alum, finally spray dried into a dry powder. This formulation is used to elicit long lasting higher antibody titers than alum adsorbed antigen or admixture of polymer entrapped antigen and alum.

### BACKGROUND OF THIS INVENTION

The tremendous power of particulate vaccine delivery system has only recently been recognized and employed strategically in vaccine design. The entrapment of antigen in particles clearly alters its acquisition and processing by antigen presenting cells and ensuing adaptive immunity. The ability of antigen to elicit immune response is called “immunogenicity”. The current vaccine formulation elicit either humoral or cell mediated immune response depending on the mode of delivery and adjuvant used.

Although the new antigens offer advantages in specificity and safety, they are in many cases weakly immunogenic. This lack of immunogenicity has created an acute need to identify pharmaceutically acceptable delivery systems or adjuvants.

More than 60 years ago aluminum hydroxide adsorbed allergens extract were introduced for depot vaccination, showing improved stimulatory as well as reduced anaphylactic properties however, there are a number of disadvantages of using alum including increased sensitivity to alum and local granuloma formation at injection sites. In addition, exposure of vaccine containing aluminum containing adjuvants to temperatures outside of the recommended storage ranges (especially exposure to freezing temperatures) puts the vaccines at risk. Although the mechanism of action is not fully understood, it is likely that surface area, surface charge, and morphology of the adjuvant are important factors dictating the immune response to antigens adsorbed onto these adjuvants (Hem and White 1984).

In various prior art, lyophilization (freeze drying) method has been used to improve long term stability of various protein preparations. However, when vaccines formulated with aluminum-salt adjuvants are processed in an attempt to improve stability through freezing and lyophilization, a loss of potency is often reported. Previous studies have suggested that a freeze-dried vaccine product containing adjuvant cannot be produced due to aggregation of the adjuvant particles. (Diminsky et al., 1999; Maa et al., 2003).

Reference may be made to U.S. Pat. No. 5,902,565 wherein an immediate-release preparation comprising an immunogen adsorbed to an aluminum adjuvant has been disclosed. It provides a method for the production of an immediate-release vaccine preparation by forming an aqueous suspension of aluminium salt-adsorbed immunogen, and subsequently spray-drying said suspension. The alum adsorbed Ag were added to the polymer (page 4, example 1).

Reference may be made to US 2004/0213798 A1 wherein a gel forming free flowing powder suitable to use as a vaccine is described. The aluminum adjuvant salt having antigen adsorbed thereon and the saccharide, amino acid or salt thereof and colloidal substance are suspended in water.

The microencapsulation of proteins in biodegradable polymers is now well recognized for controlled-release vaccines requiring only a single administration.

Reference may be made to United States Patent Application 20100112078 wherein vaccine composition comprising an effective amount of antigen or a nucleic acid encoding antigen, encapsulated in polymeric particles has been described however, it does not disclose anything about the spray dried alum formulation of the present invention.

Reference may be made to Baras et al, Elsevier: Vaccine 18(200)1495-1505, wherein microencapsulation of an antigen by spray-drying preserved its crucial characteristics required to generate an effective humoral immune response after a single-dose administration has been demonstrated.

Reference may be made to “Stabilization of alum-adjuvanted vaccine dry powder formulations” by Yuh-Fun Maa et al, Journal of Pharmaceutical Sciences, Volume 92 Issue 2, Pages 319-332 wherein the mechanism of alum gel coagulation upon freezing and drying and its relationship to vaccine potency loss is elucidated.

It has been also reported in the prior art that it is important to preserve the physical, chemical, and biological properties of the protein while producing formulations of therapeutic proteins. In contrast to lower molecular weight drugs, proteins typically have large globular structures, including secondary, tertiary, and in some cases, quaternary structural features that are important for biological activity.

One way to stabilize drugs is to embed them in biodegradable polymeric microparticles (Refer: Maulding (1987), J. Controlled Release 6:167-176; Smith et al. (1990), Advanced Drug Delivery Reviews 4:343-357; Holland et al. (1986), J. Controlled Release4:155-180; Lewis et al. (1990), Biodegradable Polymers as Drug Delivery Systems, pp. 1-41, Dekker, New York.)

Studies using microparticles made from homo- and co-polymers of lactic and glycolic acid (PLGA polymers) have also shown that these polymers hydrolyze to acid monomers (Maulding (1987), J. Controlled Release 6:167-176; Smith et al. (1990), Advanced Drug Delivery Reviews 4:343-357; Cower et al. (1985), Methods in Enzymology 112:101-116) and are chemically unreactive under the conditions used to prepare the microparticles. Such polymers can be produced in a range of molecular weights and monomer ratios which allows adjustment of the drug release rate to the particular application. PLGA polymers are non-immunogenic and non-toxic. These properties led to the selection of a PLGA polymer for use in the depot formulation of the luteinizing hormone releasing hormone (LHRH) agonist luprolide (Sanders et al. (1986), J. Pharm. Sci. 75:356-360; Ogawa et al. (1988), Chem. Pharm. Bull. 5:1095-1103; Ogawa et al. (1988), Chem. Pharm. Bull. 36:2576-2581). Johnson et al. (1997), Pharmaceutical Research 14:730-735, stabilized recombinant human growth hormone by forming a zinc-protein complex and encapsulated the complex in the solid state into PLGA microparticles (see also PCT Application No. PCT/US95/05511, Publication No. WO 95/29664).

Despite their advantages for stabilizing proteins, the administration of polymer-based drug formulations can be problematic.

In spite of extensive research on immunogenicity of polymer particle entrapped antigens, very often the immune response generated from particulate antigen is lower than that achieved with the multiple dose of alum adsorbed immunization. A further improvements in immune response comparable and even better than alum (aluminum hydroxide gel) adsorbed antigens have been reported by immunization with admixture of alum and particle entrapped antigen such as Diphtheria toxoid and Hepatitis B surface antigen (Singh et al., 1998; Gupta et al., 1998; Shi et al., 2002).

In addition to the use of additional adjuvants, hydrophobicity of the particles, size and load of entrapped antigen also influence its immunogenicity (Katare et al., 2003; Katare et al., 2005; Katare and Panda, 2006). Enhanced immune response have also been reported with a combination of alum and biodegradable nanoparticles containing tetanus toxoid indicating co-operative adjuvant effect of biodegradable nanoparticles in combination with alum (Raghuvanshi et al., 2001; Kanchan and Panda, 2007).

It has also been reported that microparticles alone generate low levels of IgG1 but presence of alum improves IgG1 levels, a known Th2 response (antibody response) indicator (Katare and Panda, 2006). This indicates that alum does play an important role in improving the immunogenicity of polymer particle entrapped antigens. Further improvements in vaccine formulation can be achieved by make a single preparation of polymer particles, antigen and alum. As lyophilized alum looses its adjuvant activity, it is imperative to use alternative procedure to make dry powder formulation.

EP1792628 patent application provides vaccine compositions comprising an oil in water emulsion optionally with 3 De-O-acylated monophosphoryl lipid A and QS21.

EP1905449 patent application relates to adjuvant compositions which is suitable to be used in vaccines. In particular, the adjuvant compositions of this invention comprise a saponin and an immunostimulatory oligonucleotide, optionally with a carrier. Methods of treating an individual susceptible to or suffering from a disease by the administration of the vaccines of the present invention are also provided.

WO/1998/015287 application relates to a vaccine composition comprising alum, an antigen, an immunologically active saponin fraction and a sterol.

WO/2002/080965 application relates to new, advantageous DTP-based combination vaccine formulations, and concomitantly administered combination vaccine kits. Methods of administration of these vaccines and kits are also provided.

EP1297844 patent application comprises N. meningitidis outer membrane vesicles enriched with antigenic components. The composition is suitable for use in vaccines and for treatment of Gram negative bacterial infection, particularly meningococcal infection, demonstrating a broad spectrum of protection to a number of different bacterial pathogens. Methods for preparation of these compositions and their uses in vaccination against disease are also provided.

U.S. Pat. No. 7,709,010 patent relates to pharmaceutical compositions comprising virus-like particles (VLPs) of HPV, said VLPs adsorbed to an aluminum adjuvant, and an ISCOM-type adjuvant comprising a saponin, cholesterol, and a phospholipid. Another aspect of this patent provides multi-dose HPV vaccine formulations comprising HPV VLPs and an antimicrobial preservative selected from the group consisting of: m-cresol, phenol and benzyl alcohol.

U.S. Pat. No. 6,544,518 patent refers to adjuvant compositions which are suitable to be used in vaccines. In particular, the adjuvant compositions of the present invention comprises a saponin and an immunostimulatory oligonucleotide, optionally with a carrier.

U.S. Pat. No. 6,251,678 patent describes human papilloma virus (HPV) vaccine formulations exhibit enhanced long-term stability. Formulation components can include: virus-like particles (VLPs) absorbed onto aluminum, a salt, non-ionic surfactant, and a buffer. Additional formulations also contain a polymeric polyanionic stabilizer and a salt either in the presence or absence buffering agents and nonionic detergent.

Poly(lactide-co-glycolide) (PLGA) and poly(lactide) (PLA) polymer particles used for vaccine delivery improve the immunogenicity of the entrapped antigens (Langer et al., 1997; Lofthouse, 2002; O'Hagan and Singh, 2003).

It has been reported that aluminium salt adjuvants have an optimal size range of less than 10 μm for adjuvant action, mainly because it is thought that antigen uptake by macrophages is an important determinant of adjuvant effectiveness [32]. Earlier reports from Nygaard et al. suggest that, adjuvants with smaller particle size distribution would be most immunogenic [33].

Alum the most widely used adjuvant for vaccine is mostly available in liquid form making cold chain mandatory for the preservation. Lyophilized alum looses its adjuvant activity thus not suitable for making solid doses based alum formulation. The present invention involved co-entrapment of alum and antigen in biodegradable polymer particles which is spray dried to give rise to dry powder formulation. This formulation elicit long lasting antibody titers from single dose application and holds promise for the development of novel vaccine formulation. The invention is linked to the earlier patents on polymer particle based vaccine formulation developed by National Institute of Immunology (Indian Patent no. 199589).

Thus, the Applicant submits that there is a need to develop a formulation which obviates the drawback of the formulation described in the prior art and provides an enhanced immunogenicity. There remains a need in the art for further improvements in vaccine formulation which can be achieved by making single preparation of polymer particles, antigen and alum. Making polymer particle and alum as single powder formulation will not only reduces the extra processing during immunization but will help in improving the immunogenicity from a single powder formulation.

### OBJECTIVE OF THE INVENTION

The main object of the invention is to provide an effective dry powder vaccine formulation that increases the immune response in the host.

Yet another object of the invention is to provide a method for preparing the vaccine formulation in dry powder form comprising of antigen, alum and biodegradable polymer particles.

Yet another object of the invention is to provide a spray drying method for preparing alum.

Yet another object of invention is to provide vaccine formulation in dry powder form having the capacity to elicit long lasting antibody response from a single dose application

### SUMMARY OF THE INVENTION

Accordingly, the present invention relates to a novel effective dry powder vaccine formulation that increases the immune response in the host. The formulation comprises of an antigen entrapped into a polymer particle, coated with novel alum and finally spray dried into a dry powder. This formulation is used to elicit long lasting higher antibody titers than alum adsorbed antigen or admixture of polymer entrapped antigen and alum.

### BRIEF DESCRIPTION OF THE ACCOMPANYING DRAWINGS

FIG. 1 depicts the formulation strategies for preparing microparticles using spray drying. Emulsions at various steps of emulsification were separately spray dried to produce particles of different characteristics

FIG. 2: depicts the Flow chart describing the formulation strategies adopted in co-entrapping alum and PspA in PLA microparticles.

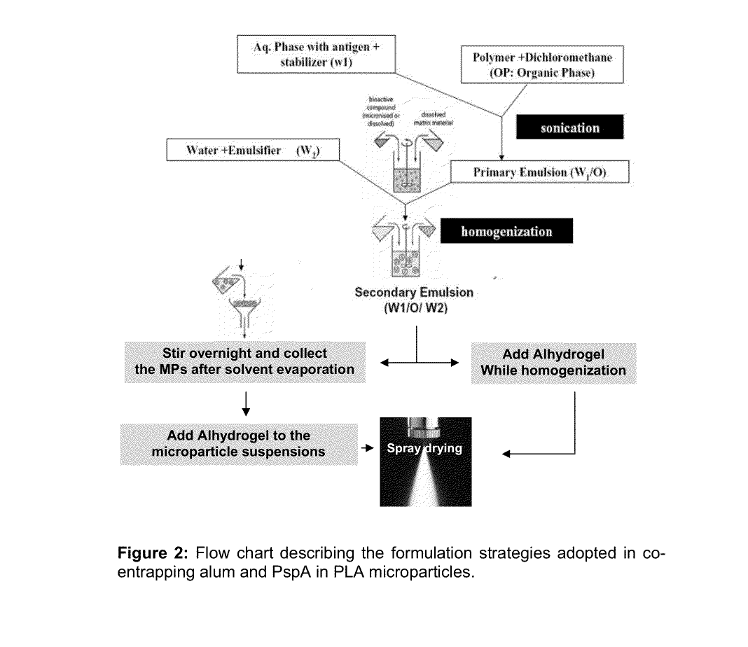

FIG. 3: depicts the particle size distribution of microparticle formulations made using

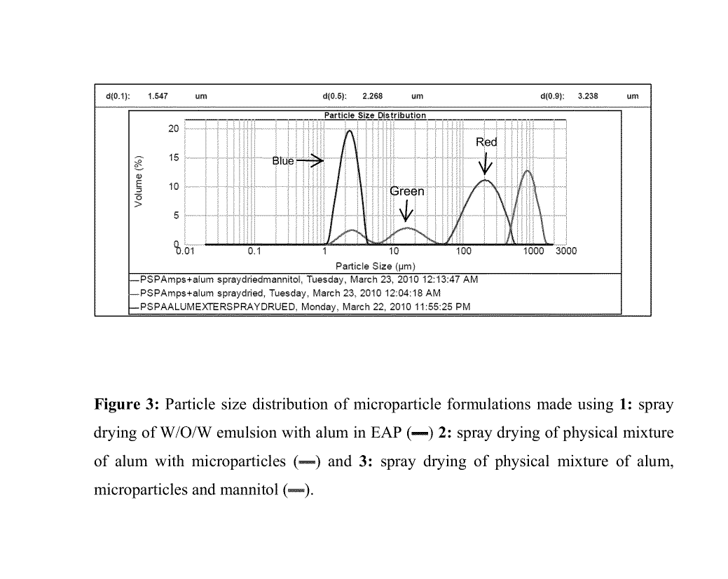

a. 1: spray drying of W/O/W emulsion with alum in EAP (—)

b. 2: spray drying of physical mixture of alum with microparticles (—) and

c. 3: spray drying of physical mixture of alum, microparticles and mannitol (—).

FIG. 4: Microscopic images of spray dried microparticles adsorbed with alum. A1, A2, and A3 & A4: SEM images of microparticles adsorbed with alum at different magnifications, formed with spray drying of W/O/W emulsion with alum in EAP B1 & B2: SEM images of aggregated microparticles after spray drying with alum as a physical mixture

FIG. 5: depicts the Microscopic images of spray dried alum powders (A: fluorescent microscopic image of spray dried alum with FITC-BSA, B: Light microscopic image of spray dried alum C: overlay A & B, D: SEM image of spray dried alum)

FIG. 6: depicts the EDX spectrum (A) and corresponding SEM image (B) of PLA microparticles without alum (control).

FIG. 7: depicts the EDX spectrum (A) and corresponding SEM image (B) of PLA microparticles surface adsorbed with alum.

FIG. 8: depicts the Elemental mapping image of alum coated microparticles. RED spots indicate the presence of aluminium

FIG. 9: depicts the Antibody response in BALB/c mice immunized with PspA entrapped PLA microparticles along with alum as adjuvant. Animals were immunized with spray dried PLA particles encapsulating PspA (—▴—) and spray dried PLA particles co-entrapping alum (—▾—) as a single dose. PLA microparticles made using conventional double emulsion solvent evaporation (——), co-lyophilized dry powders of alum with particles (—▪—) and particles physically mixed with alhydrogel (—♦—) were used as control. Serum anti-PspA IgG antibody titers were expressed as the O. D. value measured at 490 nm.

FIG. 10: (A) size analysis of DT particles entrapped in alum (B) SEM of DT entrapped alum particles.

FIG. 11: Antibody titer from alum microparticles entrapping DT.

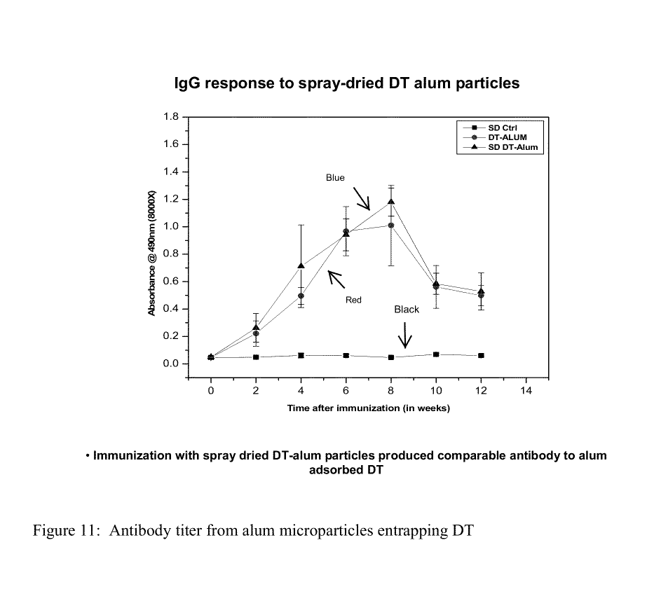

FIG. 12. Size analysis of TT entrapped alum particles.

FIG. 13. Antibody titers from tetanus toxoid entrapped in alum particles.

### DETAILED DESCRIPTION OF THE INVENTION

The present invention relates to a novel effective dry powder vaccine formulation that increases the immune response in the host. The formulation comprises of an antigen entrapped into a polymer particle, coated with novel alum and finally spray dried into a dry powder. This formulation is used to elicit long lasting higher antibody titers than alum adsorbed antigen or admixture of polymer entrapped antigen and alum.

For protective immunity, polymer particle based vaccine delivery systems provide a viable alternative to multi-dose immunization schedule for many infectious diseases where neutralizing antibody titers. Particles, particularly made from poly lactide-co-glycolide (PLGA) or PLA, not only work as a delivery system but also provide adjuvant activity. These polymeric particulate delivery systems have the capacity to promote presentation of the antigen by both MHC class I (MHC I) and MHC class II (MHC II) pathway and thus can activate both humoral and cellular response. Efficient targeting of particulate antigen to the APCs has been reported as a major factor contributing towards the generation of immune response, which requires that the particle size should be between 1-10 μm. The improved immunogenicity of polymer particle entrapped antigen is associated with the continuous delivery of the antigen in to APC and its interaction with macrophages, DC etc for antigen presentation. Immunogenicity of many antigens has been further improved while using alum as an additional adjuvant along with polymer particles.

The kit according to this invention comprises compositions or vaccines in relation to the method of immunization proposed. The kit according to the invention therefore comprises a container containing various containers containing the compositions or vaccines and advantageously, and optionally, an explanatory brochure including useful information for administration of the said compositions or vaccines.

When introducing elements disclosed herein, the articles “a”, “an”, “the”, and “said” are intended to mean that there are one or more of the elements unless the context dictates otherwise. For example, the term “a compound” and “at least one compound” may include a plurality of compounds, including mixtures thereof.

The terms “comprising”, “having”, “including” are intended to be open-ended and mean that there may be additional elements other than the listed elements. As is understood by the skilled person, administration of a vaccine can be done in a variety of manners. For example, administration may be done intramuscularly, subcutaneously, intravenously, intranasally, intradermaly, intrabursally, in ovo, ocularly, orally, intra-tracheally or intra-bronchially, as well as combinations of such modalities. The dose of the vaccine may vary with the size of the intended vaccination subject.

### ABBREVIATIONS USED

Alum—Aluminium hydroxide
EAP—external aqueous phase
IAEC—Institute Animal Ethics Committee
IAP—internal aqueous phase
MSA—mouse serum albumin
OP—organic phase
PEG—polyethylene glycol
PLA—Poly (D, L-Lactide)
PspA—Pneumococcal Surface antigen A
PVA—poly vinyl alcohol
PVP—polyvinyl pyrrolidone
RSA—rate serum albumin
SEM—Scanning Electron Microscope
(W/O)—Water/oil
(W/O/W)—water/oil/water
(W₁/O/W₂)—water-in-oil-in-water

The major embodiment of this invention is a to provide a effective dry powder vaccine formulation for eliciting the long lasting higher antibody titre comprising an antigen entrapped into a biodegradable polymer co entrapped with alum.

Another embodiment of this invention is the formulation is in the form of effective dry free flowing micro particle powder.

Still another embodiment of this invention is the alum is selected from Aluminium hydroxide gel and Aluminium phosphate gel.

Yet another embodiment of this invention is the alum used is about 2% w/v to the polymer particles.

Preferred embodiment of this invention is alum is coated evenly to polymer particle surface to reduce the aggregation of particles.

Another preferred embodiment of this invention is the polymer is biodegradable poly (D,L-Lactide).

Yet another preferred embodiment of this invention is the antigen is selected from a group comprising of recombinant pneumococcal surface antigen Psp A, tetanus toxoid, etc.

Still another preferred embodiment of this invention is the alum is spray dried to microparticles size in the range of 1-10 μm.

Yet another preferred embodiment of this invention is the alum is spray dried to microparticles size in the range of 2-8 μm.

Yet another preferred embodiment of this invention is the formulation is re-dispersible with uniform size and shape.

Most preferred embodiment of this invention is the method for producing the effective dry powder vaccine comprising the steps of;
a. Mixing aquase phase Ag, Stabilizer (w1), polymer and organic phase (OP or O);
b. Sonication of the mixture of step (a) to get primary emulsion (W₁/O),
c. Mixing water and emulsifier with the Primary emulsion of Step (b),
d. Homogenization of the mixer obtained by step (c) to get secondary Emulsion (W₁/O/W₂) water-in-oil-in-water,
e. Evaporating the solvent from the secondary emulsion of step (d) by stirring overnight to produce the microparticles, and
f. Adding Alhydrogel to the microparticles of step (e) for lyophilizing/spray drying to collect the product;

Optionally,
a. Adding Alhydrogel to in step (d) while homogenization, and
b. Lyophilizing/Spray drying the homogenized emulsion of step (g) to elute the product.

Another preferred embodiment of this invention is the formulation obtained is stored at temperature in the range of 2-8° C.

Yet another preferred embodiment of this invention is the organic phase (OP or O) is 50 mg/ml PLA (45 KDa) solution in dichloromethane.

Still another preferred embodiment of this invention is the internal aqueous phase (IAP) comprises of protein antigen, and excipient like rat serum albumin (RSA) or mouse serum albumin (MSA) (2.5% w/v), sodium bicarbonate (NaHCO₃) (2% w/v), and sucrose (10% w/v).

Still another preferred embodiment of this invention is the external aqueous phase (EAP) comprises of polyvinyl alcohol (PVA) (1% w/v) and sucrose (10% w/v) as excipient.

Yet another embodiment of this invention is a kit comprising;
a. dry powder formulation of this invention,
b. a diluent or excipient, and
c. an instruction manual

One of the major problems associated with admixture of polymer particles and alum is that the particles are solid where as alum need to be stored under low temperature for adjuvant activity.

Lyophilization of alum lead to its loss of adjuvant activity. Making polymer particle and alum as single solid powder formulation will not only reduces the extra processing during immunization but will help in improving the immunogenicity from a single powder formulation. This will make the polymer containing alum formulation more thermos abler as alum is in dried state. Candidate vaccine have the poor generation of memory antibody response after immunization.

The other novel application of the process is to spray dry the alum alone and make it particles in the range of 2-8 microns. These particles are stable and have higher antigen adsorption capacity thus provide better adjuvant action upon immunization. The novelty of this invention has been observed with recombinant PspA antigen.

The invention is now illustrated by various examples and accompanying drawings, which are not meant to limit the scope of the invention in any manner. All embodiments that may be obvious to a skilled person would fall within the scope of the present invention.

### Example 1

### Formulation of Poly (D, L-lactide) (PLA) Particles Entrapping PspA Using Double Emulsion Solvent Evaporation

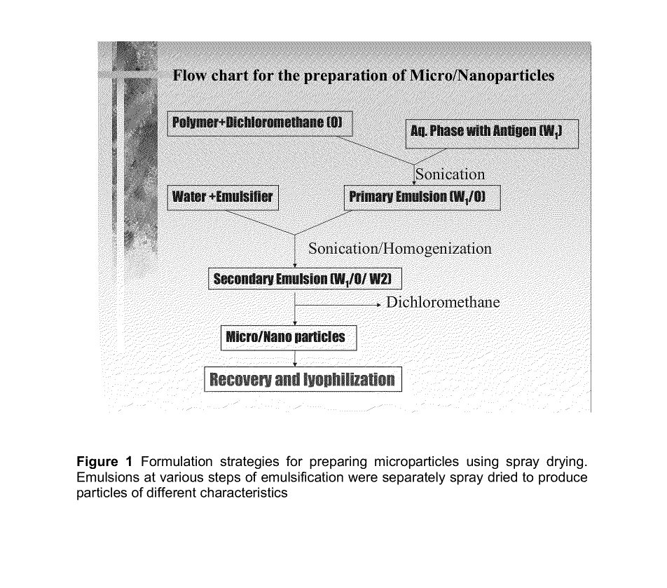

PLA polymer particles were prepared using water-in-oil-in-water (W₁/O/W₂) double emulsion solvent evaporation method [FIG. 1, 2]. Briefly, primary emulsion between internal aqueous phase (IAP or W₁) containing the antigen and organic phase (OP or O) (50 mg/ml PLA (45 KDa) solution in dichloromethane) was prepared by sonication using probe KE-76 (SONOPULS HD 2200, Ultrasonic Homogenizer, Bandelin, Germany) (40% duty cycle, 20% power output, 1 minute) on ice. In addition to the protein antigen (10-30 mg/ml), excipients like rat serum albumin (RSA) or mouse serum albumin (MSA) (2.5% w/v), sodium bicarbonate (NaHCO₃) (2% w/v), and sucrose (10% w/v); were incorporated into IAP. EAP comprised of polyvinyl alcohol (PVA) (1% w/v) and sucrose (10% w/v) as excipients. Resulting primary emulsion was added drop wise to external aqueous phase (EAP or W₂) and homogenized (5,000-15,000 rpm for 10 minutes) using a homogenizer (POLYTRON® PT-3100, Kinematica AG, Switzerland) on ice. The resulting emulsion was kept overnight stirring at room temperature under sterile conditions and the particles were collected by centrifugation at 15,000 rpm for 20 minutes, washed three times with ice-cold Milli Q water (15,000 rpm for 20 minutes each) and lyophilized to get free flowing powder. Polymer particle were then stored at 4° C. in a desiccator.

### Example 2

### Preparation of PspA Loaded Microparticles Using Spray Drying

Polylactide particles encapsulating PspA were prepared using spray drying. Briefly, primary emulsion between internal aqueous phase (IAP or W₁) containing the antigen and organic phase (OP or O) (PLA (45 KDa) solution in dichloromethane) was prepared by sonication using probe KE-76 (SONOPULS HD 2200, Ultrasonic Homogenizer, Bandelin, Germany) (40% duty cycle, 20% power output, 1 minute) on ice. In addition to the protein antigen (10-30 mg/ml), IAP comprised of excipients like rat serum albumin (RSA) or mouse serum albumin (MSA) (2.5% w/v), sodium bicarbonate (NaHCO₃) (2% w/v), and sucrose (10% w/v); whereas EAP comprised of an emulsion stabilizer like polyvinyl alcohol (PVA) or polyvinyl pyrrolidone (PVP-K30) or polyethylene glycol (PEG) (1-5% w/v) and a polyhydroxy compound like sucrose/lactose/mannitol/sorbitol as excipients. Details of each optimized formulations are described in following sections. Resulting primary emulsion was added drop wise to external aqueous phase (EAP or W₂) and homogenized (5,000-15,000 rpm for 10 minutes) using a homogenizer (POLYTRON® PT-3100, Kinematica AG, Switzerland) on ice as per the requirement. The resultant emulsions were then spray dried in a co-current spray system (Twin-cyclon Laboratory Spray Drier: LU-227Advanced Model, Labultima, Mumbai, India), with nozzle size of 0.5 mm, two fluid spray nozzle) at different stages of emulsion process. The emulsions were spray dried either at primary emulsion stage (W/O) or at secondary emulsion stage (W/O/W) stage as shown in the flowchart in FIG. 1. Final formulations for immunizations were made using the optimized parameters. The final formulations were hermetically sealed and stored at 2-8° C.

### Example 3

### Preparation of PspA Loaded Spray Dried Microparticles Co-Entrapping Alum (Alhydrogel™)

To co-entrap antigen (PspA: Pneumococcal surface antigen A) and alum in PLA particles, different formulation strategies were optimized. PLA particles prepared using conventional double emulsion solvent evaporation was mixed with alum and were lyophilized to make dry powder formulations. The role of pre-freezing on the stability of alum was evaluated by varying the pre-freezing processes like shelf freezing and freezing in liquid nitrogen before lyophilization. Spray drying was also optimized to make re-dispersible dry powder formulations using the process parameters. To facilitate in situ coating of alum during spray drying, Alhydrogel™ was added to the external aqueous phase of W/O/W double emulsion during homogenization and the emulsion was directly spray dried to produce free flowing dry powders. The role of different excipients like polyols in preventing the aggregation of alum was also studied. Alhydrogel™ suspensions in lyophilized form as well as spray dried form were also prepared to be used as control formulations in the study.

### Example 4

### Immunization Studies Using Spray Dried Microparticle Formulations Entrapping PspA

All animal experiments were carried out in out bred female Wistar rats (8 weeks old) or male BALB/c mice (6-8 weeks old, inbred mice). Animals were maintained according to the guidelines established by the Institute Animal Ethics Committee (IAEC) of the National Institute of Immunology, New Delhi, India. Immunizations were carried out in sterile saline (pH 7.4). Immunogenicity of PLA particles containing PspA was evaluated in Wistar rats (six female out bred Wistar rats per group). PLA microparticles with size range of 2-8 μm (average size=2.16 μm) entrapping 5 μg PspA were used for immunization as a single dose. Required dose of particles were weighed and suspended in normal saline just before immunization. Immunization of admixture of particles and alum were carried out by adding 25 μL of alum (Aluminium hydroxide gel, 2% w/v) to the required dose of polymer particles per animal. Alum adsorbed soluble recombinant PspA and PspA microparticle formulations developed using conventional double emulsion were used as controls. Rats were immunized intramuscularly with 5 μg of PspA encapsulated in microparticles. Animals were later challenged with 1 μg soluble PspA in saline after twelve months. Animals were bled at different time intervals through retro-orbital plexus and serum antibody titers were determined by ELISA.

### Example 5

### Formulation and Evaluation of PLA Microparticles Entrapping PspA and Alum (Alhydrogel™) Using Spray Drying

PLA particles co-entrapping both PspA and alum were prepared both by solvent evaporation method and spray drying method. When microparticles were formulated using spray drying of W/O/W secondary emulsion, the alum coated evenly to the particle surface and this prevented alum induced aggregation of particles as well as aggregation of alum itself. This resulted in narrow size distribution of polymeric particles. Thus adding alum to the EAP while homogenization to make W/O/W emulsion and to directly spray dry this emulsion was found to be the ideal formulation strategy for making PLA microparticles co-entrapping antigen and alum. In order to confirm the role of aggregation in size distribution, the particles were analyzed using scanning electron microscopy.

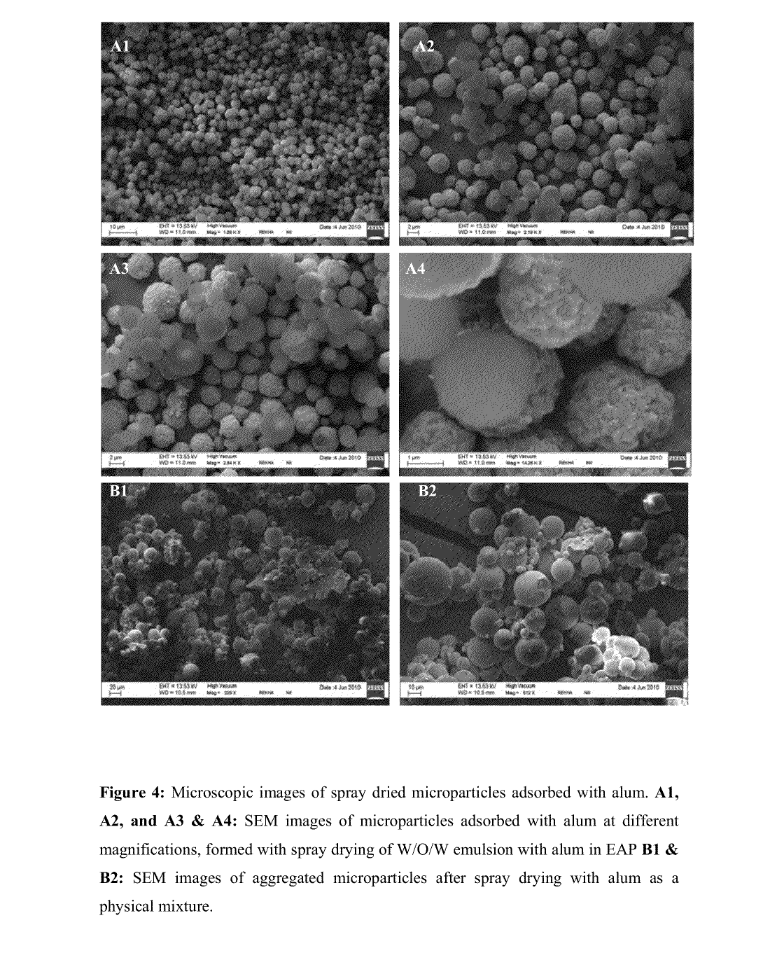

The particles prepared by direct spray drying of W/O/W secondary emulsion with alum in EAP had a corrugated rough surface structure possibly due to surface adsorption of alum (FIG. 4: A1, A2, A3, A4 shows alum adsorbed polymer particles at different magnifications).

More over these particles had uniform size distribution and the particles were not aggregated. Where as formulations prepared by spray drying of preformed particles with alum was smooth in surface morphology due to absence of alum coating and were highly aggregated (FIG. 4: B1 and B2).

Thus when physical mixture of alum and microparticles were spray dried, alum induced aggregation of particles as well as alum formed large agglomerates. This resulted in broad particle size distribution. When alum is added to the EAP of W/O/W alum got in situ adsorbed evenly while the particles are formed after evaporation of solvent. Thus even adsorption of alum avoids agglomeration of alum as well as alum induced aggregation. This was not observed when physical mixture of alum and microparticles were employed for development of formulation. Both lyophilization as well as spray drying resulted in segregation of alum and particles. This prevented adsorption of alum to the particle surface.

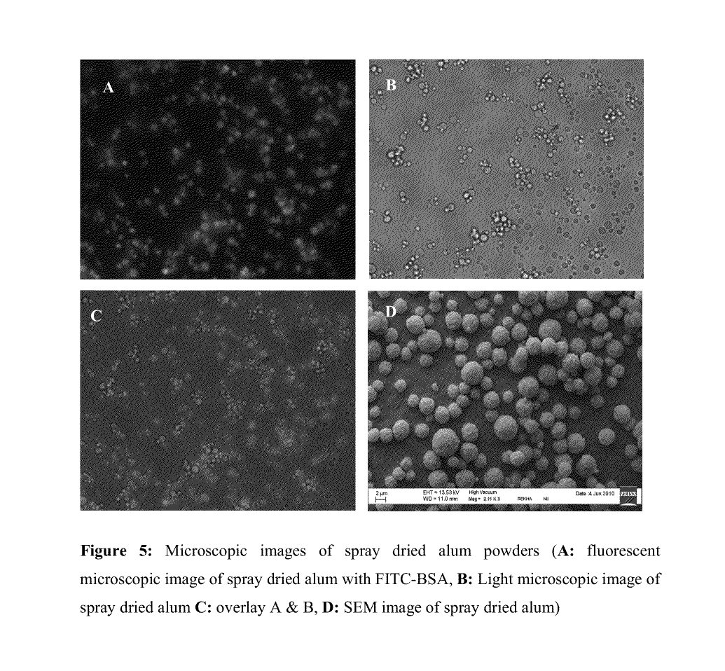

Using spray drying, alum forms re-dispersible powders with uniform size distribution and shape (FIG. 5). Spray drying alum resulted in particles with large specific surface area and uniform size distribution which aid re-dispersion of the dry powder. These particles entrapped the antigen FITC-BSA uniformly in the core.

Since alum on spray drying has an inherent tendency to form uniform sized particles, addition of alum to the W/O/W emulsion resulted in uniform coating of alum on surface of the particles. This prevented aggregation and resulted in narrow size distribution. These results indicated that alum can be delivered along with antigen entrapped PLA particles as a supplementary adjuvant. The results open up new possibilities in the field of antigen delivery using microparticles. Since alum alone can form microparticles with spray drying, this can be used for delivering the antigen.

### Example 6

### Elemental Mapping Analysis and Energy Dispersive X-Ray Spectroscopy (EDX) Coupled with SEM to Localize the Presence of Alum on the Microparticles

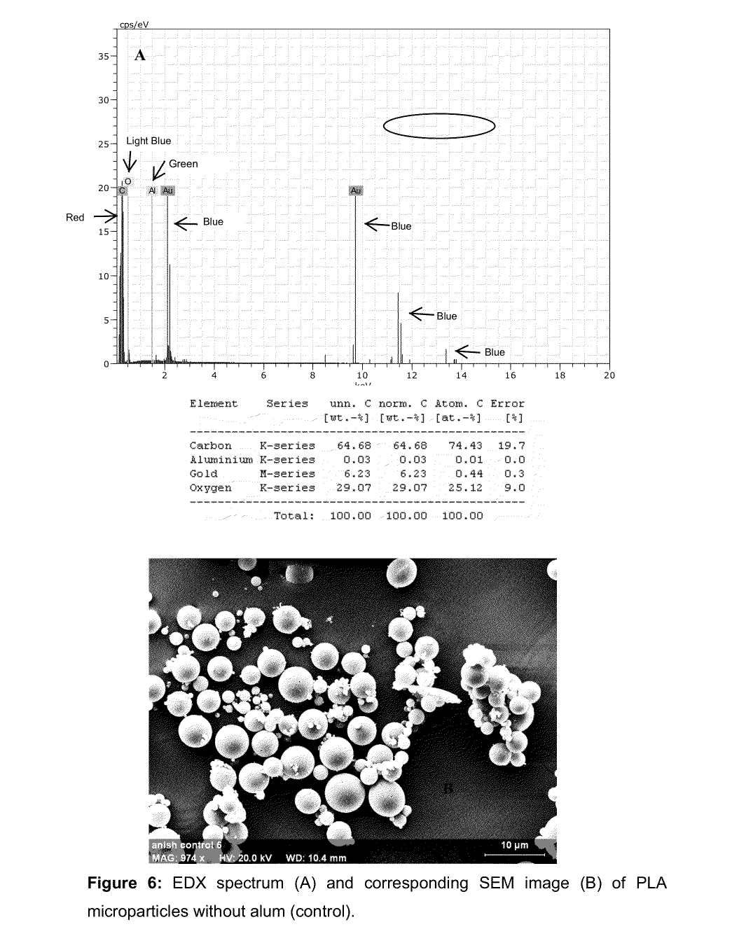

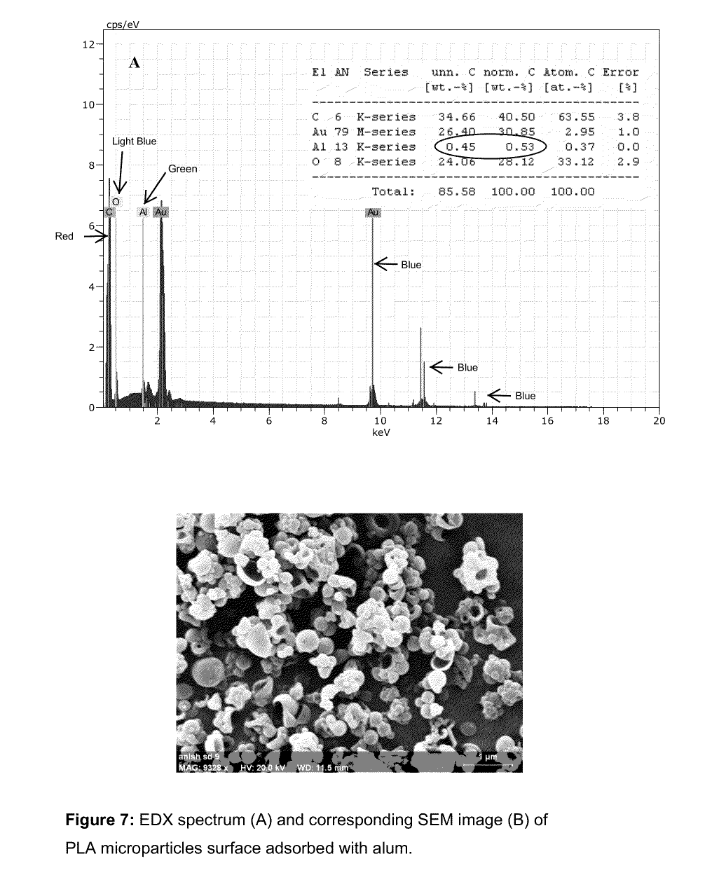

The presence of alum adsorption on the particle surface was confirmed using EDX coupled with SEM. Microparticles were spread evenly on an iron stub using a thin needle, coated with sputter coater and imaged using SEM. The aluminium specific spectral lines were recorded. However microparticles coated with alum produced aluminium specific spectral lines equivalent to an intensity of 0.53 wt. % (FIG. 7). Whereas in the blank uncoated particles used as control these spectral lines were almost absent showing an average intensity of 0.03 wt % (FIG. 6). The presence of aluminium spectral lines are due to the presence of alhydrogel attached to the particle surface. This confirmed that the spray drying W/O/W emulsion by adding alum in EAP helps in uniform coating of alum on the particle surface and delivers alum along with antigens.

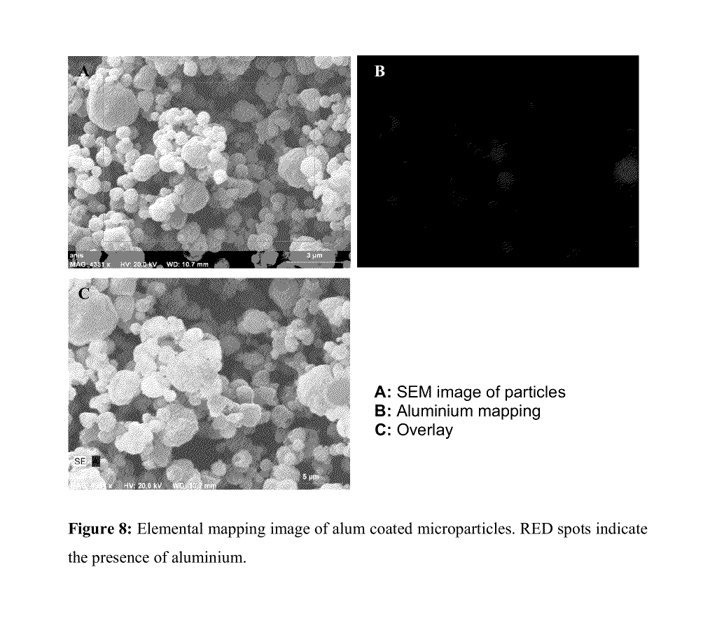

To further visualize the presence of alum on the particle surface, elemental mapping was performed. FIG. 8 describes the results of elemental mapping studies. The red spots on the particles indicated the presence of alum. The images clearly confirmed the uniform distribution of alum on the surface of the PLA microparticles.

The presence of alum on the surface of these particles can delay the distribution of released antigen from the site of injection. Alum could adsorb the released antigen from particles and retain them for longer time. More over, since alum effectively interacts with innate immune system, presence of alum on the particle surface might improve the immunological properties of these particles. The co-delivery of alum along with microparticles as surface attached dry powder formulation is also a better strategy in pharmaceutical point of view considering the low costs associated with storage and transport of liquid alhydrogel suspensions. Since dry powders have better stability than liquid formulations, this might improve the stability of alhydrogel. Thus in the vaccine delivery context, this formulation has immense potentials to translate in to a successful clinical product.

### Example 7

### Immunization Studies Using Spray Dried Microparticle Formulations Co-Entrapping Antigen and Alum

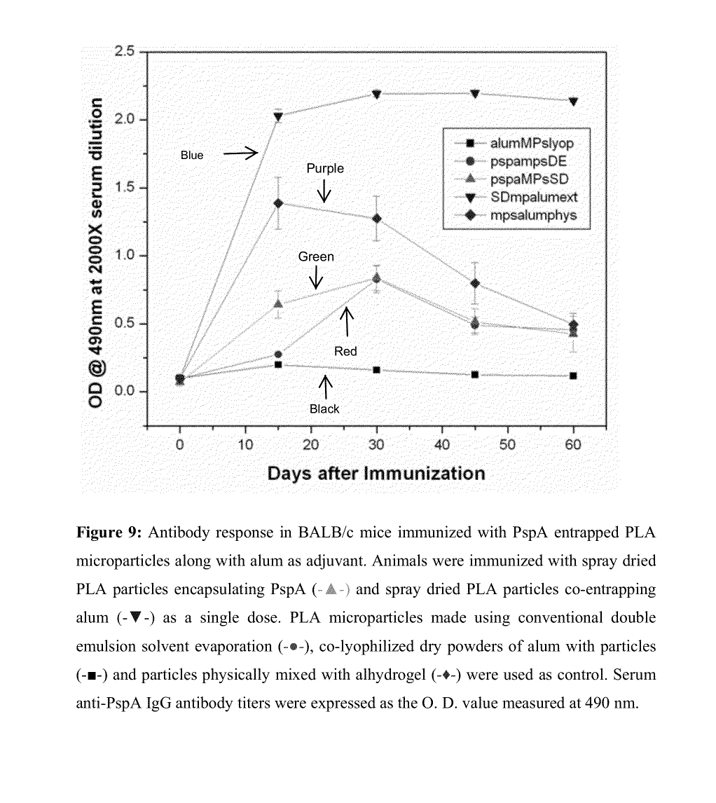

Spray dried microparticle formulations entrapping PspA were immunized intramuscularly to BALB/c mouse. Experimental group contained six animals per group. Particles formulated using conventional double emulsion solvent evaporation was used as the control. To evaluate the effect of alum, a physical mixture of alhydrogel was administered along with PspA entrapped polymer particles. In case of spray drying, microparticles co-entrapping alum and recombinant PspA were used for the studies. Particles equivalents to 5 μg PspA were re-suspended in normal saline for injection before immunization. The details of the formulation used for immunization studies are shown in the Table 1. Serums from immunized animals were collected periodically. The anti PspA antibody response was evaluated using ELISA and represented as OD value plotted against days after immunization (FIG. 9). As evident from the figure, alum worked synergistically with microparticles in improving the antibody response to PspA. Immunizing with physical mixture of microparticles and alhydrogel resulted in higher antibody response than immunizing with PLA particles alone.

Table 1: Details of formulations used for immunization studies. (DE-MPs microparticles made using conventional double emulsion solvent evaporation and SD-MPs microparticles made using spray drying; All animals were immunized through intra muscular route after re-suspending the particles in normal saline. For immunization with physical mixture of both alum and MPs both were mixed before injection).

| | | | Bese of | Amount MPs |
|---|---|---|---|---|
| | | Antigen | PspA/ | used for 8 |
| Group No. | Formulations | load | animal | animals |
| Group-I | DE-MPs | 3.67 μg/mg | 5 μg | 10.89 mg/1 ml |
| | | | | normal saline |
| Group-II | SD-MPs | 3.73 μg/mg | 5 μg | 10.72 mg/1 ml |
| | | | | normal saline |
| Group-Ill | DE-MPs + Alum | 3.67 μg/mg | 5 μg | 10.89 mg/1 ml |
| | Physical Mixture | | | normal saline |
| Group-IV | DE-MPs + Alum | 3.67 μg/mg | 5 μg | 10.89 mg |
| | co-lyophilized | | | MPs + 120 μl |
| | | | | alum/1 ml |
| | | | | normal saline |
| Group-V | SD-MPs + Alum | 0.834 μg/mg | 5 μg | 47.96 mg/1 ml |
| | co-spray dried | | | normal saline |

But this effect was not there when animals were immunized with dry powders made using co-lyophilization of alum and microparticles. This was due to the aggregation of alum which resulted in increase in size distribution.

Therefore, it was concluded that retaining the original PSD of the adjuvant particles following processing would result in the most effective vaccine. Lyophilization induced agglomeration of alum and aggregation of particles resulted in higher particle size. This led to failure of these formulations in inducing a strong antibody response. But when alum was coated uniformly on the surface of the PLA microparticles using spray drying, the particles induced a strong anti-PspA antibody response. This could be due to better presentation of antigen to the APCs by the microparticles. Also since alum is present on the surface of the particles it can interact with innate immune system and create a pro-inflammatory milieu at the site of injection. It can also delay the distribution of released antigen from the site of injection [27]. These effects improved the immunogenicity of PspA entrapped in PLA particles.

### Example 8

### Immunization Studies Using Spray Dried Microparticle Formulations Co-Entrapping Diphtheria Toxoid and Alum

Alum (Adju-phos, 2% Aluminium Phosphate Gel Adjuvant; Brenntag Biosector, Denmark) and Water was mixed in a ratio of 2:1, 1 ml of the antigen solution [DT (15.8 mg/ml)] was added to this mixture. This mixture was kept on stirring and spray dried to obtain free flowing particles. Size and zeta potential of the particles was analyzed using Malvern mastersizer hydro 2000S and zetasizer respectively. Size of the particles was found to be 3-8 μm. Surface morphology was analyzed using a scanning electron microscope (SEM)—JEOL (JSM 6100, Tokyo, Japan)—after coating the particle surface with gold-palladium over an aluminium stub.

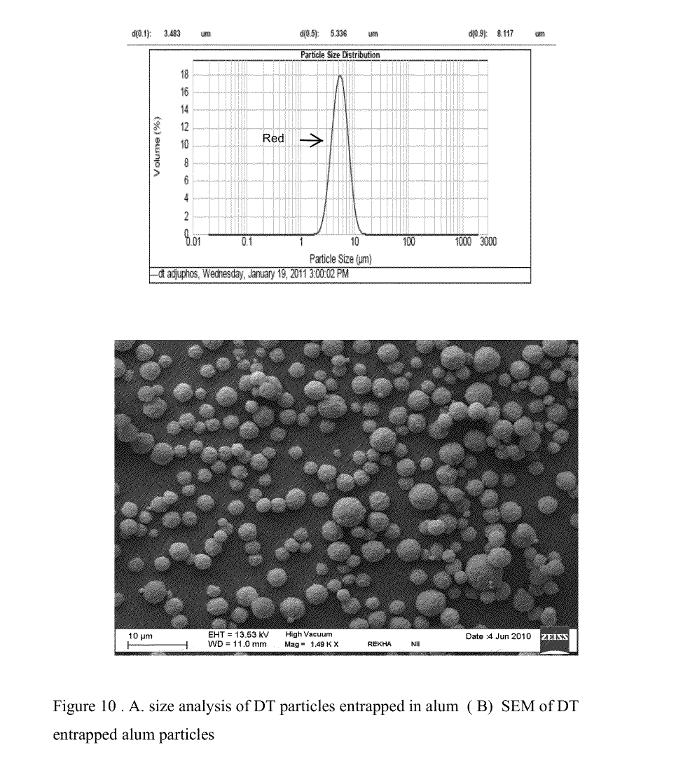

To measure the protein content of particles, accurately weighed particles were dissolved in 0.2 M NaOH to solubilize the particles. Protein was then precipitated using TCA precipitation. Precipitated protein was dissolved in 1% SDS solution and estimated using micro BCA assay. Alum particles entrapping tetanus toxoid were prepared by spray drying. The average size of the particles were of 2-8 um and the load of DT on alum particle was around 13.09 ug/mg. These particles were immunized intramuscularly to mice and antibody titers were estimated by ELISA (2). The antibody titers is presented in FIG. 10. It was observed that single point immunization DT entrapped in alum particles (SD DT-Alum) elicited comparable titers as observed with DT adsorbed on liquid alum preparation. This suggested that the adjuvant effect of alum is not lowered upon spray drying. More importantly it leads to the development of solid formulation which will be more stable and easy to handle. Spray dried formulation of alum particle entrapping DT thus become more stable without loosing the adjuvant effect of alum. A single formulation of antigen and alum as dry powder provide number of advantages in comparison to the liquid formulate of alum adsorbed DT.

### Example 9

### Immunization Studies Using Spray Dried Microparticle Formulations Co-Entrapping Tetanus Toxoid and Alum

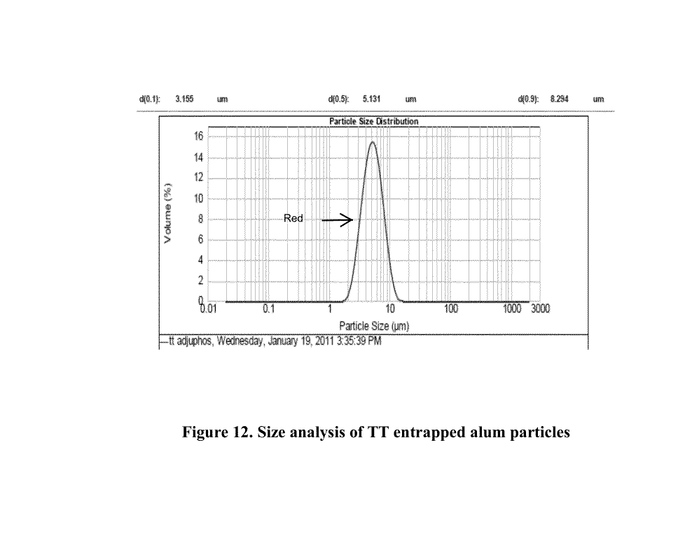

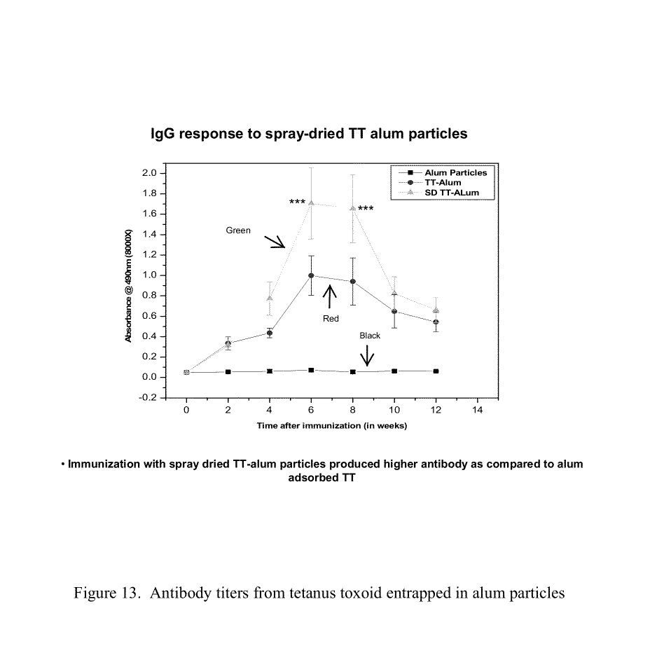

Alum (Adju-phos, 2% Aluminium Phosphate Gel Adjuvant; Brenntag Biosector, Denmark) and Water was mixed in a ratio of 2:1, 1 ml of the antigen solution TT (23.67 mg/ml) was added to this mixture. This mixture was kept on stirring and spray dried to obtain free flowing particles. The size analysis of the spray dried TT [particle entrapped in alum is given in FIG. 12. The average size of TT entrapped alum particles was around 2-6 micron and the load of TT per mg of particles was around 19.47 ug/mg. These particle were immunized intramuscularly and antibody titers were estimated as described earlier for TT (FIG. 13). It was observe that like TT entrapped in alum particles elicited antibody titre much better than that observed with alum adsorbed TT. This signifies the improved immunogenicity of the spray dried alum particle based formulation. Like DT, entrapments of TT in alum particles and its spray dried formulation stabilized the product and leads to solid phase formulation of TT vaccine. Spray drying also did not reduce the adjuvant action of alum. Spray dried formulation of vaccine in alum particle thus provide multiple advantages including improving its stability.

### Example 10

### In the Form of Kit(s)

The formulation is made as a medical kit, which includes an additional component(s) of various kinds if needed. The additional component for example is preferably selected from the group consisting of: an injection needle, disinfectant, a drape, a knife, likewise and is not limited to a particular kind According to the medical kit, it is possible to more readily and safely use/administer the formulation of the present invention.

The medical kit of the present embodiment preferably includes a liquid that can be used as an injection solution. The kit composition may be formulated as a pourable liquid like solution, controlled release shields of patches, suspension, ointment, gels and so on.

The kit according to this invention comprises formulation or composition in relation to the method proposed. The kit therefore comprises of various containers containing the compositions, excipients and optionally, an explanatory brochure including useful information for using said compositions or patches.

### Advantages

### Some of the Advantages of this Formulation in Comparison to Other Alum Formulation are

An effective dry powder vaccine formulation that increases the immune response by several folds in the host than the existing methods.

No deep storage or special storage measures are needed for this formulation.

Alum adsorbs the released antigen from particles and retains them for longer time. The presence of alum on the surface of these particles delay the distribution of released antigen from the site of injection.

Elicits long lasting antibody response from a single dose application.

Alum on spray drying has an inherent tendency to form uniform sized particles, addition of alum to the W/O/W emulsion resulted in uniform coating of alum on surface of the particles. This prevented aggregation and resulted in narrow size distribution.

Alum effectively interacts with innate immune system and improves the immunological properties of these particles.

The co-delivery of alum along with microparticles as surface attached dry powder formulation is also a better strategy in pharmaceutical point of view considering the low costs associated with storage and transport of liquid alhydrogel suspensions.

Since dry powders have better stability than liquid formulations, this might improve the stability of alhydrogel. Thus in the vaccine delivery context, this formulation has immense potentials to translate in to a successful clinical product

1. A vaccine formulation for eliciting the long lasting higher antibody titre comprising an antigen entrapped into a biodegradable polymer co entrapped with alum.

2. The formulation as claimed in claim 1 is in the form of effective dry free flowing micro particle powder.

3. The formulation as claimed in claim 1 wherein said alum is selected from Aluminium hydroxide gel and Aluminium phosphate gel.

4. The formulation as claimed in claim 1 wherein said alum used is about 2% w/v to polymer particles.

5. The formulation as claimed in claim 1 wherein said alum is coated evenly to polymer particle surface to reduce the aggregation of particles.

6. The formulation as claimed in claim 1 wherein said polymer is biodegradable poly (D,L-Lactide).

7. The formulation as claimed in claim 1 wherein said antigen is selected from a group comprising of recombinant pneumococcal surface antigen Psp A, tetanus toxoid, etc.

8. The formulation as claimed in claim 1 wherein the alum is spray dried to microparticles size in the range of 1-10 μm.

9. The formulation as claimed in claim 8 wherein said alum is spray dried to microparticles size in the range of 2-8 μm.

10. The formulation as claimed in claim 1 wherein the formulation is re-dispersible with uniform size and shape.

11. A method for producing the effective dry powder vaccine formulation of claim 1 comprising the steps of;
a. Mixing aquase phase Ag, Stabilizer (w1), polymer and organic phase (OP or O);
b. Sonication of the mixture of step (a) to get primary emulsion (W₁/0),
c. Mixing water and emulsifier with the Primary emulsion of Step (b),
d. Homogenization of the mixer obtained by step (c) to get secondary Emulsion (W₁/O/W₂) water-in-oil-in-water,
e. Evaporating the solvent from the secondary emulsion of step (d) by stirring overnight to produce the microparticles, and
f. Adding Alhydrogel to the microparticles of step (e) for lyophilizing/spray drying to collect the product
Optionally,
g. Adding Alhydrogel to in step (d) while homogenization, and
h. Lyophilizing/Spray drying the homogenized emulsion of step (g) to elute the product.

12. The method as claimed in claim 11 wherein the formulation obtained is stored at temperature in the range of 2-8° C.

13. The method as claimed in claim 11, wherein said organic phase (OP or O) is 50 mg/ml PLA (45 KDa) solution in dichloromethane.

14. The method as claimed in claim 11, wherein the internal aqueous phase (IAP) comprises of protein antigen, and excipients like rat serum albumin (RSA) or mouse serum albumin (MSA) (2.5% w/v), sodium bicarbonate (NaHCO₃) (2% w/v), and sucrose (10% w/v).

15. The method as claimed in claim 11, wherein the external aqueous phase (EAP) comprises of polyvinyl alcohol (PVA) (1% w/v) and sucrose (10% w/v) as excipients.

16. A kit comprising;
a. dry powder formulation of claim 1
b. a diluent or excipient, and
c. an instruction manual.
